(19) United States
(12) Patent Application Publication
(10) Pub. No.: US 2017/0196899 A1
(43) Pub. Date: Jul. 13, 2017

(54) STABLE PHARMACEUTICAL COMPOSITIONS

(71) Applicant: GALENICUM HEALTH S.L., Barcelona (ES)

(72) Inventors: Sergio ARROYO HIDALGO, Barcelona (ES); Mireia PLADEVALL ROSÉS, Barcelona (ES)

(21) Appl. No.: 15/471,641

(22) Filed: Mar. 28, 2017

Related U.S. Application Data
(63) Continuation of application No. 14/897,788, filed on Dec. 11, 2015.

(30) Foreign Application Priority Data
Nov. 12, 2013 (EP) 13192572.9
Feb. 12, 2014 (EP) 14154931.1
Apr. 11, 2014 (EP) 14164490.6

(51) Int. Cl. A61K 31/65; A61K 38/05; A61K 9/08; A61K 9/19; A61K 9/16; A61K 47/18; A61K 47/02

(52) U.S. Cl. CPC A61K 31/65; A61K 47/183; A61K 38/05; A61K 47/02; A61K 9/19; A61K 9/1682; A61K 9/08

(57) ABSTRACT

The present invention relates to a stable pharmaceutical composition comprising tigecycline and arginine, to a process for the manufacture of said pharmaceutical composition as well as to its use in the treatment of bacterial infections.

The present invention relates to a stable pharmaceutical composition comprising tigecycline and trehalose, to a process for the manufacture of said pharmaceutical composition as well as to the its use in the treatment of bacterial infections.

### STATE OF THE ART

The need for new antibacterial agents is greater than ever because of the emergence of multidrug resistance in common pathogens and the rapid emergence of new infections. Bacterial resistance is associated with treatment failures, increased mortality, and increased costs. Methicillin-resistant Staphylococcus aureus (S. aureus), glycopeptide-intermediate S. aureus and glycopeptide-resistant enterococci are of particular concern. In addition, resistance of gram-negative bacteria to all approved antimicrobials is emerging. Despite the need for new antimicrobial agents, the development of these agents is declining.

Tigecycline is also known as N-[(5aR,6aS,7S,9Z,10aS)-9-[amino(hydroxy)methylidene]-4,7-bis(dimethylamino)-1,10a,12-trihydroxy-8,10,11-trioxo-5,5a,6,6a,7,8,9,10,10a,11-decahydrotetracen-2-yl]-2-(tert-butylamino)acetamide, or as 9-t-butyl glycylamido minocycline. The empirical formula of tigecycline is C29H39N508 and the compound has a molecular weight of 585.65 g/mol. The structural formula of tigecycline is (I):

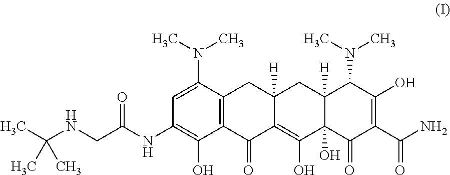 [SMILES: CN(C)c1cc(NC(=O)CNC(C)(C)C)c(O)c2c1C[C@H]1C[C@H]3[C@H](N(C)C)C(O)=C(C(N)=O)C(=O)[C@@]3(O)C(O)=C1C2=O]

Tigecycline is a member of the glycylcycline class of antimicrobial agents. It carries a glycylamido moiety attached to the 9-position of minocycline. As a bacteriostatic agent, tigecycline inhibits the growth of multiple resistant gram-positive, gram-negative, anaerobic, and atypical bacteria, including methicillin-resistant S. aureus and extended-spectrum β-lactamase producers. Tigecycline works by blocking the bacteria's ribosomes, the parts of the cell where new proteins are made. By blocking the production of new proteins, the bacteria cannot multiply and they eventually die.

EP0536515 discloses 7-substituted-9-(substituted amino)-6-demethyl-6-deoxy tetracycline compounds having activity against a wide spectrum of organisms including organisms which are resistant to tetracyclines. U.S. Pat. No. 5,675,030 discloses a method for the selective extraction of tigecycline. WO2006099258 relates to tigecycline compositions comprising lactose and an acid or buffer. Tygacil® is used to treat adults with complicated infections of the skin and soft tissue (the tissue below the skin), but not foot infections in people with diabetes. It is also used to treat complicated infections in the abdomen. “Complicated” means that the infection is difficult to treat. Tygacil® contains 50 mg of tigecycline as active substance and is presented as powder for solution for infusion. Tygacil® needs to be stored below 25° C. and, once reconstituted and diluted for infusion, it should be used immediately. Therefore, there is a need for stable pharmaceutical composition comprising tigecycline. Moreover, it would be desirable to provide compositions having a good stability and a long shelf life.

### DESCRIPTION OF THE INVENTION

The present invention provides stable pharmaceutical compositions of glycylcyclines and arginine. It has been found that arginine can stabilize glycylcyclines in a particular weight ratio, so that pharmaceutical compositions comprising essentially a glycylcycline such as tigecycline and arginine, preferably L-arginine, are more resistant to degradation.

The present invention also provides a stable pharmaceutical composition comprising tigecycline or a pharmaceutically acceptable salt thereof. It has been found that trehalose stabilizes tigecycline or a pharmaceutically acceptable salt thereof and that injectable pharmaceutical compositions comprising tigecycline or a pharmaceutically acceptable salt thereof and trehalose are surprisingly stable against degradation.

Tigecycline is easily degraded by oxidation and epimerization, so it is necessary to provide compositions comprising said active agent which are stable. The present invention provides a highly stable composition both in lyophilised form and as a solution after reconstitution of the lyophilised cake or powder.

The present invention therefore provides a pharmaceutical composition comprising tigecycline or a pharmaceutically acceptable salt thereof, and trehalose. Advantageously, lyophilised preparations are stable, can be stored and are easily reconstituted. Moreover, lyophilised preparations may be kept sterile and essentially free of insoluble matter.

In a first aspect, the present invention provides a pharmaceutical composition comprising an active agent and trehalose, wherein the active agent is tigecycline or a pharmaceutically acceptable salt thereof.

In a second aspect, the present invention provides a lyophilised cake or lyophilised powder obtainable by lyophilisation of the pharmaceutical composition of the first aspect, wherein said lyophilised cake or powder is suitable for reconstitution to form a liquid composition for parenteral, preferably intravenous, administration.

In a third aspect, the present invention provides a pharmaceutical composition comprising the lyophilised cake or powder of the second aspect reconstituted in a sterile aqueous solution.

In a fourth aspect, the present invention provides a process for the manufacture of a pharmaceutical composition of the first, second and third aspects, comprising at least the following step:
i) preparing a solution of trehalose and the active agent.

The term “pharmaceutically acceptable” indicates that the substance or composition must be compatible chemically and/or toxicologically, with the other ingredients comprising a pharmaceutical composition, the mammal being treated therewith, and/or the route of administration of the composition.

In a preferred embodiment of the pharmaceutical composition of the first aspect, the weight ratio active agent:trehalose is between 10:1 to 1:10, preferably 2:1 to 1:4. Preferably, the weight ratio active agent:trehalose is between 3:2 to 1:3. More preferably, it is between 1:1 to 1:2.

Trehalose is also known as a-D-glucopyranosyl-(1→1)-α-D-glucopyranoside or as mycose or tremalose. Its empirical formula is C12H22O11 and it has a molecular weight of 342.3 g/mol.

The term “active ingredient” refers to a therapeutically active compound, as well as any prodrugs thereof and pharmaceutically acceptable salts, hydrates and solvates of the compound and the prodrugs.

In a preferred embodiment of the pharmaceutical composition of the first aspect, the pharmaceutical composition further comprises at least one suitable amino acid or a salt thereof. Preferably, the suitable amino acid is selected from glycine, proline, cysteine, aspartic acid, serine, threonine, alanine, valine, isoleucine, leucine, methionine, phenylalanine, tyrosine, tryptophan, arginine, histidine, lysine, a salt thereof and a mixture thereof. More preferably, the suitable amino acid is selected from arginine, histidine, lysine, a salt thereof and a mixture thereof. Even more preferably, the suitable amino acid is selected from arginine, histidine, a salt thereof or a mixture thereof. In a preferred embodiment, the suitable amino acid is arginine or a salt thereof. Preferably, the suitable amino acid consists of arginine or a salt thereof. In a preferred embodiment of the pharmaceutical composition of the first aspect, the weight ratio active agent:suitable amino acid or suitable amino acids is between 2:1 to 1:2, preferably is between 3:2 to 2:3, more preferably is between 6:5 to 5:6.

The term “suitable amino acid” as used herein refers to an amino acid which is compatible chemically and/or toxicologically with tigecycline and which can be safely administered parenterally, preferably intravenously. Suitable amino acids are glycine, proline, cysteine, aspartic acid, serine, threonine, alanine, valine, isoleucine, leucine, methionine, phenylalanine, tyrosine, tryptophan, acetyltryptophan, arginine, histidine, lysine, and lysine acetate. Preferred amino acids are arginine and histidine. The most preferred amino acid is arginine or a salt thereof. Also, derivatives of said amino acids such as acetyltryptophan or lysine acetate may be used.

In a preferred embodiment of the pharmaceutical composition of the first aspect, the pharmaceutical composition further comprises a bulking agent. Preferably, the bulking agent is selected from the group consisting of a saccharide such as glucose, mannose, galactose, ribose, fructose, sucrose, lactose, raffinose, mannitol, inositol, sorbitol; an oligomer such as cyclodextrin; a polymer such as glycogen, starch, dextran, chitosan, hyaluronate, polyvinylpyrrolidone; a protein such as gelatin or serum albumin; and a mixture thereof. More preferably, the bulking agent is selected from lactose, galactose, mannitol and a mixture thereof. Even more preferably, the bulking agent is mannitol. In another preferred embodiment, the bulking agent is lactose. In a preferred embodiment, the weight ratio active agent:bulking agent or bulking agents is between 5:1 to 1:5, preferably is between 3:1 to 1:3, more preferably is between 2:1 to 1:2. The term “bulking agent” refers to a pharmaceutically acceptable excipient and which provides bulk to the formulation such that when unit dosage amounts of the solution are lyophilised in containers, such as sterile vials, the freeze-dried residue will be clearly discernible. Acceptable bulking agents include, but are not limited to, carbohydrates such as simple sugars such as dextrose, ribose, fructose and the like, alcohol sugars such as mannitol, inositol and sorbitol, disaccharides including sucrose and lactose, naturally occurring polymers such as starch, dextrans, chitosan, hyaluronate, proteins (e.g., gelatin and serum albumin) and glycogen, and synthetic monomers and polymers. Bulking agents for use in the present invention preferably also act as osmolytes (i.e., aid in making the liquid form of the formulation isotonic with normal human serum).

In a preferred embodiment of the pharmaceutical composition of the first aspect, the pharmaceutical composition comprises between 25 and 75 mg of active agent by unit dose. Preferably, the pharmaceutical composition comprises between 45 and 55 mg, preferably about 53 mg of active agent by unit dose. As used herein, the term “unit dose” or “unit dosage” refers to a physically discrete unit that contains a predetermined quantity of active ingredient calculated to produce a desired therapeutic effect. The unit dose or unit dosage may be in the form of a vial, tablet, capsule, sachet, etc. referred to herein as a “unit dosage form”.

In a preferred embodiment of the pharmaceutical composition of the first aspect, the pharmaceutical composition further comprises a pH modifying agent. Preferably, the pH modifying agent is an acid. Preferably, the acid is hydrochloric acid. The term “pH modifying agent” as used herein refers to an excipient capable of modifying the pH of the composition. The pH of the composition is that of the solution comprising tigecycline or a pharmaceutically acceptable salt thereof and trehalose, before lyophilisation or after reconstitution of the lyophilised cake or powder.

In a preferred embodiment of the pharmaceutical composition of the first aspect, the composition is in form of a solution and is suitable to be lyophilised. Preferably, the solution is an aqueous solution. In a preferred embodiment, the pH of the solution ranges from 3.0 to 8.0. More preferably, the pH of the composition is between 4.0 and 7.0. Even more preferably, the pH of the composition is between 4.5 and 6.0. In a preferred embodiment, the concentration of the active agent is between 5 and 55 mg/ml, preferably about 26.5 mg/ml. In a more preferred embodiment, said concentration is about 53 mg/mi.

In a preferred embodiment of the pharmaceutical composition of the first aspect, the composition is in the form a lyophilised cake or powder, wherein said lyophilised cake or powder is suitable for reconstitution to form a liquid composition for parenteral, preferably intravenous, administration. Preferably, the water content of the lyophilised cake or powder is below 3.0% by weight of the total amount of the lyophilised cake or powder. More preferably, it is below 2.0% by weight of the total amount of the lyophilised cake or powder. Even more preferably, it is between 0.1% and 1.0% by weight of the total amount of the lyophilised cake or powder.

Lyophilisation, also called freeze-drying, can be used to prepare dry pharmaceutical formulations to achieve commercially viable shelf lives. The process comprises three steps: freezing, primary drying, and secondary drying. As water freezes in the first step, the dissolved components in the formulation remain in the residual liquid, a phase termed the freeze-concentrate. At the point of maximal ice formation, the freeze-concentrate solidifies between the ice crystals that make up the lattice. Under appropriate lyophilisation conditions, the ice is removed by sublimation during primary drying, leaving the remaining freeze-concentrate in the same physical and chemical structure as when the ice was present. Residual water in the freeze-concentrate is removed in the secondary drying step. Lyophilisation works by freezing the material and then reducing the surrounding pressure to allow the frozen water in the material to sublimate directly from the solid phase to the gas phase. The lyophilisate can be rehydrated (reconstituted) to its original form for injection. This is done much more quickly and easily because the lyophilisation process leaves microscopic pores. The pores are created by the ice crystals that sublimate, leaving gaps or pores in their place. When a lyophilisate is sealed to prevent the reabsorption of moisture, it may be stored and shipped at room temperature without refrigeration, and be protected against spoilage for many years, thus increasing its shelf life. Preservation is possible because the greatly reduced water content inhibits the action of microorganisms and enzymes that would normally spoil or degrade the substance. The lyophilisate resulting from the lyophilisation process can be in form of cake or powder.

Once lyophilised, the pharmaceutical compositions as herein disclosed are packaged (e.g. the vials are stoppered) and ready for storing and/or shipping to end users. For use, the lyophilised cake or powder is reconstituted by adding a suitable reconstitution solution. Suitable sterile solutions for reconstitution of the lyophilisate form of the pharmaceutical compositions as herein disclosed are distilled and/or sterile water for injection, bacteriostatic water for injection optionally comprising methylparabene and/or propylparabene and/or 0.9% w/w benzyl alcohol, saline, 5% glucose solution, 5% or 10% dextrose solution, Hartmann's solution, Ringer's solution and/or Ringer's lactated solution. Preferably, saline is 0.9% sodium chloride, or 0.45% or 0.225% solution of sodium chloride. Also, a solution of 0.45% sodium chloride and 2.5% dextrose can be used. Other suitable solutions can be 6% dextran in 5% dextrose or 6 to 10% hydroxyethyl starch solutions in sterile water for injection.

In a preferred embodiment of the pharmaceutical composition of the first aspect, the active agent is in amorphous form. In a preferred embodiment of the pharmaceutical composition of the first aspect, the active agent is in crystalline form. For manufacturing the pharmaceutical compositions as herein disclosed, crystalline tigecycline, amorphous tigecycline or a pharmaceutically acceptable tigecycline salt in amorphous or crystalline form can be used.

In a preferred embodiment of the third aspect, the sterile aqueous solution is distilled and/or sterile water for injection, bacteriostatic water for injection optionally comprising methylparabene and/or propylparabene and/or 0.9% w/w benzyl alcohol, saline, 5° A glucose solution, 5% dextrose solution, Hartmann's solution, Ringer's solution and/or Ringer's lactated solution. Preferably, the sterile aqueous solution is saline, Ringer's lactated solution or dextrose solution.

Preferably, the concentration of the active agent of the pharmaceutical compositions as herein disclosed is between 0.5 to 25 mg/ml, preferably around 10 mg/ml.

The pharmaceutical compositions of the invention are stable. The term “stable” as used herein refers to a pharmaceutical composition comprising tigecycline wherein the total content of impurities originating from the decomposition of tigecycline does not exceed 5% area, preferably 3% area, more preferably 2% area determined by liquid chromatography (HPLC) at 248 nm if such a composition is stored for 2 months at 35° C. and 65% relative humidity (RH).

In another aspect, the present invention relates to a pharmaceutical composition wherein the concentration of tigecycline epimer is between 0.05% to 5%, preferably between 0.5% to 4%, more preferably between 0.7% to 3.5%.

In a preferred embodiment, the pharmaceutical composition of the invention essentially consists of the active agent, trehalose, lactose and hydrochloric acid. Preferably, its pH is between 4.5 and 6.0.

In a preferred embodiment of the process of the fourth aspect, the solution of step (i) is prepared by (i1) preparing a solution of trehalose and adjusting its pH below 7, preferably between 4.5 and 6.0, and (i2), adding the active agent. Preferably, the process further comprises step (ii) adjusting the pH of the solution prepared in step (i) to between 3.0 to 8.0, preferably between 4.0 and 7.0, more preferably between 4.5 and 6.0. In a preferred embodiment, the process further comprises at least one of the following steps:
iii) filtering the solution obtained in step (ii),
iv) freezing the solution obtained in step (iii), and
v) freeze-drying the frozen solution obtained in step (iv).

Preferably, steps (i), (ii) and optionally step (iii) is performed at a temperature below 15° C. Preferably, the solution is an aqueous solution. Preferably, steps (iii), (iv) and (v) are carried out in aseptic conditions. In a preferred embodiment, step (iii) is performed into a vial. Preferably, the vial is sealed after lyophilisation under an inert atmosphere. Preferably, the inert atmosphere is nitrogen atmosphere.

In another aspect, the present invention relates to the pharmaceutical composition obtained by the process of the fourth aspect.

In another aspect, the present invention relates to a pharmaceutical batch of the pharmaceutical composition of the first, second or third aspects, or of the pharmaceutical composition obtained by the process of the fourth aspect. Preferably, the pharmaceutical batch comprises at least 150 units. More preferably, it comprises at least 2,000 units.

The term “batch” as used herein refers to a specific quantity of a drug or other material that is intended to have uniform character and quality, within specified limits, and is produced according to a single manufacturing order during the same cycle of manufacture. A batch, in the case of a drug product produced by continuous process, is a specific identified amount produced in a unit of time or quantity in a manner that assures its having uniform character and quality within specified limits (Code of Federal Regulations Title 21, Food and Drug Administration department of Health and Human Services, Subchapter C, Section 210.3 (b) (2) and (10)).

The term “pharmaceutical batch” as used herein refers to a batch as defined above of a pharmaceutical composition manufactured in accordance with the principles and guidelines of Good Manufacturing Practice (GMP) at an industrial scale and which is intended for commercialization (Directive 91/356/EEC).

The pharmaceutical composition may be manufactured at laboratory scale, not necessarily following GMP and not intended for commercialization. The pharmaceutical composition may also be manufactured for validation, following GMP. A batch of a pharmaceutical composition which is manufactured for validation is called “pilot batch”.

Each pharmaceutical batch of finished product must fulfil the regulatory requirements of the corresponding Medicine Agency before being released for sale or supply, such as impurities thresholds and stability data.

The term “uniform” as used herein refers to the content of the active ingredient in the vials of a pharmaceutical batch has to be homogeneous. According to the FDA criteria, uniformity is considered as achieving 90-110% potency of the theoretical strength with a relative standard deviation (RSD) of less than 5% for all samples (Guidance for Industry ANDA's: Blend Uniformity Analysis, published August 1999).

In a fifth aspect, the present invention relates to a vial comprising the pharmaceutical composition of the first, second or third aspects or obtained by the process of the fourth aspect, wherein said vial is a tubular glass vial. In a preferred embodiment, the volume of said vial is between 3 and 12 ml, preferably between 4 and 11 ml. Preferably, said vial is capped with a rubber stopper and a seal.

In another aspect, the present invention relates to a cardboard box with a patient information leaflet comprising at least one unit of the pharmaceutical composition of the first, second or third aspects, or of the pharmaceutical composition obtained by the process of the fourth aspect or at least one vial of the fifth aspect.

In another aspect, the present invention relates to a process for the manufacture of a pharmaceutical composition comprising an active agent and trehalose, wherein the active agent is tigecycline or a pharmaceutically acceptable salt thereof, or for the manufacture of a pharmaceutical batch of said pharmaceutical composition, wherein the process comprises the following steps:

i) preparing a test pharmaceutical composition comprising the active agent and trehalose;

ii) checking the stability of the test pharmaceutical composition of step (i), preferably the stability after at least one day at 40° C. and 75% relative humidity; and

iii) manufacturing a pharmaceutical composition comprising the active agent and trehalose or a pharmaceutical batch of said pharmaceutical composition, by the same process used to prepare the test pharmaceutical composition in step (i) provided that the test composition is stable; and

iv) optionally, packaging the stable pharmaceutical composition or the pharmaceutical batch manufactured in step (iii), preferably in vials.

In another aspect, the present invention relates to a pharmaceutical batch of the pharmaceutical composition as disclosed herein, validated by a process comprising the following steps:

i) manufacturing the pharmaceutical batch;

ii) checking the tigecycline epimer content; and

iii) validating the batch only if the content is between 0.05% to 5%, preferably between 0.5% to 4%, more preferably between 0.7% to 3.5%.

Another aspect of the present invention is the use of the pharmaceutical composition as disclosed herein for the manufacture of a medicament, preferably an intravenous medicament, for the treatment of an infectious disease, preferably of complicated skin and soft tissue infections, or complicated intra-abdominal infections.

Another aspect relates to the pharmaceutical composition of the first, second or third aspects, or of the pharmaceutical composition obtained by the process of the fourth aspect, or the pharmaceutical batch as herein disclosed, for use in the treatment of an infectious disease.

Another aspect relates to the pharmaceutical composition of the first, second or third aspects, or of the pharmaceutical composition obtained by the process of the fourth aspect, or the pharmaceutical batch as herein disclosed, for use in the treatment of complicated skin and soft tissue infections, or complicated intra-abdominal infections.

Another aspect relates to the pharmaceutical composition of the first, second or third aspects, or of the pharmaceutical composition obtained by the process of the fourth aspect, or the pharmaceutical batch as herein disclosed, for use in the treatment of complicated skin and soft tissue infections excluding diabetic foot infections, or complicated intra-abdominal infections.

A second invention relates to a pharmaceutical composition comprising an active agent and arginine or a pharmaceutically acceptable salt thereof, wherein the active agent is a glycylcycline or a pharmaceutically acceptable salt thereof and wherein the weight ratio active agent:arginine is between 2:3 and 3:1. In a preferred embodiment, the glycylcycline is tigecycline. In a preferred embodiment, the weight ratio is between 5:6 to 2:1. In a more preferred embodiment, the weight ratio is about 1:1. The best stability results were obtained for tigecycline compositions comprising 50 mg of tigecycline and 50 mg of L-arginine.

In a preferred embodiment of the second invention, the pharmaceutical composition comprises between 25 and 75 mg of active agent by unit dose. Preferably, the pharmaceutical composition comprises between 45 and 55 mg, preferably about 53 mg of active agent by unit dose. In another preferred embodiment, the pharmaceutical composition comprises 50 mg of active agent by unit dose.

In a preferred embodiment, the pharmaceutical composition consists essentially of 50 mg of active agent by unit dose and arginine. More preferably, the pharmaceutical composition consists essentially of about 50 mg of tigecycline and about 50 mg of arginine, preferably L-arginine.

In a preferred embodiment, the active agent is tigecycline or a pharmaceutically acceptable salt thereof. Preferably, the active agent is tigecycline. Tigecycline can be in crystalline or amorphous form. Preferably, the active agent is in amorphous form.

In a preferred embodiment of the pharmaceutical compositions as disclosed in the second invention, the arginine is L-arginine. In a preferred embodiment of the pharmaceutical compositions as herein disclosed, said composition does not comprise any bulking agent, only the arginine. In a preferred embodiment, the pharmaceutical composition is free of a suitable sugar.

In another preferred embodiment of the pharmaceutical compositions as disclosed in the second invention, the pharmaceutical composition comprises at least one bulking agent. Preferably, it comprises between 25 and 85% w/w of the bulking agent or bulking agents in respect of the total amount of the pharmaceutical composition. More preferably, it comprises between 30 and 80% w/w of the bulking agent or bulking agents in respect of the total amount of the pharmaceutical composition. Even more preferably, it comprises between 40 and 70% w/w of the bulking agent or bulking agents in respect of the total amount of the pharmaceutical composition. In a preferred embodiment of the pharmaceutical compositions as disclosed in the second invention, the bulking agent is selected from the group consisting of a saccharide such as glucose, mannose, galactose, ribose, fructose, trehalose, sucrose, lactose, raffinose, mannitol, inositol, sorbitol; an oligomer such as cyclodextrin; a polymer such as glycogen, starch, dextrans, chitosan, hyaluronate, polyvinylpyrrolidone; a protein such as gelatin or serum albumin; and a mixture thereof. Preferably, the bulking agent is selected from lactose, galactose, mannitol and trehalose and a mixture thereof. More preferably, the bulking agent is selected from mannitol and trehalose and a mixture thereof. Even more preferably, the bulking agent is a mixture of mannitol and trehalose.

In another preferred embodiment of the pharmaceutical compositions as disclosed in the second invention, the pharmaceutical composition further comprises a pH modifying agent or a mixture of pH modifying agents. Preferably, the pH modifying agent is an acid or a buffer. More preferably, the acid is hydrochloric acid. In case it is necessary, the pH can also be adjusted with a base such as sodium hydroxide.

In another preferred embodiment of the pharmaceutical compositions as disclosed in the second invention, the composition is in form of a solution and is suitable to be lyophilised. When the pharmaceutical composition in form of a solution is lyophilised, a lyophilisate is obtained. Said lyophilisate can be in the form of a lyophilised cake or a lyophilised powder. In a preferred embodiment, the solution is an aqueous solution. Preferably, the pH of the solution is between 3.0 to 8.0. More preferably, the pH of the composition is between 3.5 and 7.0. Even more preferably, the pH of the composition is between 4.0 and 5.0. In a preferred embodiment, the concentration of the active agent is between 5 and 55 mg/ml, preferably about 26.5 mg/ml. In another preferred embodiment, the composition is in the form of a lyophilised cake or powder, wherein said lyophilised cake or powder is suitable for reconstitution to form a liquid composition for parenteral, preferably intravenous, administration. Preferably, the water content of the lyophilised cake or powder is below 4% by weight of the total amount of the lyophilised cake or powder. More preferably, the water content of the lyophilised cake or powder is below 3% by weight of the total amount of the lyophilised cake or powder. Even more preferably, the water content of the lyophilised cake or powder is between 0.1% and 2% by weight of the total amount of the lyophilised cake or powder. In another preferred embodiment, said lyophilised cake or powder is suitable for reconstitution to form a liquid composition for parenteral, preferably intravenous, administration.

Another aspect of the second invention is the pharmaceutical composition obtainable by reconstitution of the lyophilised cake or powder of the second invention with a sterile aqueous solution. Preferably, the sterile aqueous solution is distilled and/or sterile water for injection, bacteriostatic water for injection optionally comprising methylparabene and/or propylparabene and/or 0.9% w/w benzyl alcohol, saline, 5% glucose solution, 5% dextrose solution, Hartmann's solution, Ringer's solution and/or Ringer's lactate solution. More preferably, the sterile aqueous solution is saline, Lactated Ringer's Injection solution or dextrose solution.

Another aspect of the second invention is a process for the manufacture of a pharmaceutical composition of the second invention, comprising at least the following step:
i) preparing an aqueous solution of arginine in purified water;
ii) optionally adjusting the pH of the solution prepared in step (i) to between 3.0 to 8.0, preferably between 3.5 and 7.0, more preferably between 4.0 and 5.0;
iii) dissolving the active agent in the solution obtained in step (i) or step (ii);
iv) optionally adjusting the pH of the solution prepared in step (iii) to between 3.0 to 8.0, preferably between 3.5 and 7.0, more preferably between 4.0 and 5.0;
v) optionally filtering the solution obtained in step (iii) or in step (iv);
vi) when appropriate, freezing the solution obtained in step (iii), (iv) or (v); and
vii) when appropriate, freeze drying the frozen solution obtained in step (vi).

In a preferred embodiment, nitrogen is bubbled in the solution. In a preferred embodiment, the purified water is previously bubbled with nitrogen gas to reduce the oxygen content in the water. In a preferred embodiment, the bubbling is performed until an oxygen concentration between 0.6 and 2 ppm is reached. Both the purified water and the solution comprising the arginine or the arginine and the active agent, can be bubbled with nitrogen until an oxygen concentration between 0.6 and 2 ppm. In a preferred embodiment, steps (vi) and/or (vii) are performed in a vial. In a preferred embodiment, the vial is sealed after lyophilisation under an inert atmosphere, preferably under nitrogen atmosphere.

In a preferred embodiment, the freeze drying is performed as defined in the third invention. The freeze drying of the third invention is preferred for the compositions disclosed in the first and the second inventions.

A third invention relates to a process for the manufacture of a lyophilised cake or powder comprising a glycylcycline or a pharmaceutically acceptable salt thereof as active agent and at least one pharmaceutically acceptable excipient, wherein said process comprises the freeze drying of a solution comprising a glycylcycline or a pharmaceutically acceptable salt thereof as active agent and at least one pharmaceutically acceptable excipient, wherein said freeze drying comprises the steps of freezing the solution, primary drying and secondary drying, and wherein the freeze drying takes at least 16 hours and less than 72 hours, preferably less than 48 hours, and more preferably less than 42 hours, from the start of the freezing of the solution to the end of the secondary drying.

In a preferred embodiment, the freeze drying comprises the following steps:

i) freezing the solution at a temperature below −20° C. and maintaining said temperature for at least 1 hour;

ii) primary drying the frozen solution of step (i) under vacuum and at a temperature between -20 and 10° C., and maintaining said conditions for at least 10 hours; and

iii) secondary drying the primary dried frozen solution of step (ii) under vacuum and at a temperature between 20 and 40° C., and maintaining said conditions for at least 5 hours.

The lyophilisation (freeze-drying) process as disclosed herein comprises at least three steps: freezing, primary drying, and secondary drying. The lyophilisation process as herein disclosed provides a good quality of the resulting cake in the minimum time. This process maximizes the rate of heat transfer to each vial without causing cake collapse and avoiding slower reconstitution times. The freeze drying cycle is efficient and robust without compromising the pharmaceutical quality of the product and neither other parameters such as stability and water content.

The term “primary drying” refers to heating the material at low temperature and low pressure to cause frozen “free water” to sublimate directly to vapour. The term “secondary drying”, also known as desorption drying, refers to further heating the material, so that the bound unfrozen water absorbs the heat of desorption and becomes free water, which then absorbs the heat of evaporation and becomes vapour, finally escaping from the material.

In a preferred embodiment, the freezing temperature in step (i) is between −55 and −35° C., preferably between −50 and −40° C., maintained at least 2 hours, preferably between 2 and 4 hours. In a more preferred embodiment, the temperature in step (ii) is between −15 and 10° C., maintained at least 12 hours, preferably between 15 and 30 hours. In a more preferred embodiment, the temperature in step (iii) is between 25 and 35° C., maintained at least 7 hours, preferably between 8 and 16 hours. In a more preferred embodiment, the temperature in step (i) is between −55 and −35° C., preferably between −50 and −40° C., maintained at least 2 hours, preferably between 2 and 4 hours; and the temperature in step (ii) is between −15 and 10° C., maintained at least 12 hours, preferably between 15 and 30 hours; and the temperature in step (iii) is between 25 and 35° C., maintained at least 7 hours, preferably between 8 and 16 hours.

In a preferred embodiment, the vacuum in step (ii) ranges from 0.01 to 1 mbar, preferably from 0.05 to 0.5 mbar, more preferably from 0.1 to 0.2 mbar. In a preferred embodiment, the vacuum in step (iii) ranges from 0.001 to 1 mbar, preferably from 0.004 to 0.5 mbar, more preferably from 0.005 to 0.015 mbar.

In a preferred embodiment, the temperature in step (i) is −45° C., maintained for 2 hours; the temperature in step (ii) is 0° C., and the pressure is 0.15 mbar, and these conditions are maintained for 18 hours; and in step (iii) the temperature is 30° C., the pressure is 0.01 mbar, and these conditions are maintained for 12 h.

In a most preferred embodiment, the temperature in step (i) is −45° C., maintained for 3 hours; the temperature in step (ii) is −10° C., and the pressure is 0.15 mbar, and these conditions are maintained for 24 hours; and in step (iii) the temperature is 30° C., the pressure is 0.01 mbar, and these conditions are maintained for 10 h.

In a preferred embodiment, the glycylcycline is tigecycline. In a preferred embodiment, the pharmaceutically acceptable excipient is a bulking agent. Preferably, the solution of the glycylcycline also comprises a pH adjusting excipient, preferably an acid, preferably hydrochloric acid. In another preferred embodiment, the glycylcycline is tigecycline and the solution comprises at least one of the pharmaceutically acceptable excipients selected from trehalose, arginine and lactose. Preferably, the pharmaceutically acceptable excipient is lactose. Preferably, the pharmaceutically acceptable excipient is arginine. Preferably, the pharmaceutically acceptable excipient is trehalose. In a preferred embodiment the solution is an aqueous solution.

In a preferred embodiment, the solution comprising a glycylcycline or a pharmaceutically acceptable salt thereof as active agent is the pharmaceutical composition as defined in the first or the second inventions. Preferably, the solution is an aqueous solution of tigecycline, the pharmaceutically acceptable excipient is lactose or arginine, the ratio active agent:excipient is between 1:2 and 3:1 and the volume of the solution is between 1 ml to 3 ml. In a preferred embodiment, the solution is an aqueous solution of tigecycline, the pharmaceutically acceptable excipient is lactose, the ratio active agent:excipient is 1:2, the volume of the solution is about 2 ml and the temperature of the primary drying is about 0° C. In another preferred embodiment, the solution is an aqueous solution of tigecycline, the pharmaceutically acceptable excipient is arginine, the ratio active agent:excipient is 1:1, the volume of the solution is about 2 ml and the temperature of the primary drying is about −10° C.

Another aspect of the second invention is the pharmaceutical composition obtained by the process as defined in the second invention.

Another aspect of the second invention is a pharmaceutical batch comprising at least 1,200 units of the pharmaceutical composition as defined in the second invention. Preferably, the pharmaceutical batch comprises at least 6,000 units.

Another aspect of the second invention is the pharmaceutical composition or the pharmaceutical batch of the second invention, for use in the treatment of an infectious disease. Preferably, for use in the treatment of complicated skin and soft tissue infections, or complicated intra-abdominal infections. Preferably, for use in the treatment of complicated skin and soft tissue infections excluding diabetic foot infections, or complicated intra-abdominal infections.

Another aspect of the second invention is a tubular glass vial comprising the pharmaceutical composition of the second invention. Preferably, the volume of said vial is between 3 and 12 ml, preferably between 4 and 11 ml, more preferably is a 5 ml vial or a 10 ml vial. Preferably, said vial is capped with a rubber stopper and a seal.

Another aspect of the second invention is a cardboard box with a patient information leaflet comprising at least one unit of the pharmaceutical composition of the second invention or at least one vial of the second invention.

Unless otherwise indicated, all the analysis methods are carried out according to the European Pharmacopoeia 7th edition. All percentages, parts and ratios herein disclosed are by weight unless specifically noted otherwise. As used herein, the term “about” refers preferably to a range that is +-10%, preferably +-5%, or more preferably the value with which the term is associated.

### DESCRIPTION OF THE FIGURES

FIG. 1: Amount of total impurities (%) in the lyophilised compositions prepared according to example 1a without trehalose and 1b, with trehalose, at time zero and after 9 days storage at 40° C. and 75% RH.

FIG. 2: X-ray powder diffraction pattern (XRD) of the tigecycline used for the preparation of the compositions of example 1.

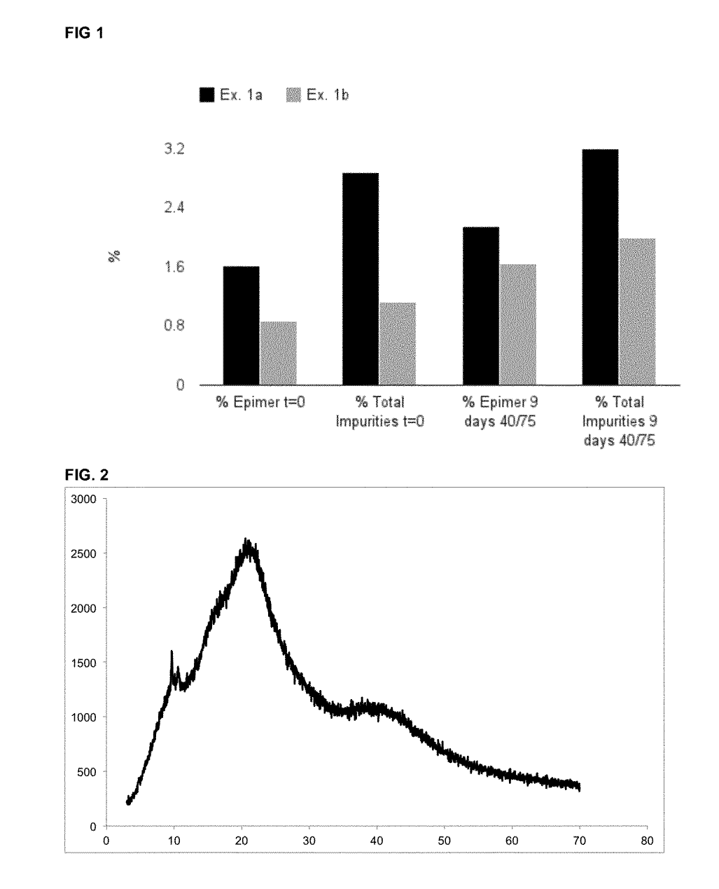

### EXAMPLES

The following examples illustrate various embodiments of the invention and are not intended to limit the invention in any way:

### Example 1

### Preparation of Stable Tigecycline Compositions

A tigecycline composition was prepared first by dissolving trehalose and any other excipient in water for injection. The solution was stirred for about 30 minutes until complete dissolution of the excipient(s). The pH of the solution was then adjusted to between 4.5 and 5.5 with HCl 1N and it was kept at a temperature between 2 and 10° C. To said solution, tigecycline was added. The pH of the solution was adjusted again to between 4.5 and 6.0 with HCl 1N, and the volume of the solution was adjusted by adding the required amount of water for injection.

The solution was filtered through a 0.22 m pore size syringe filter. The solution was thereafter transferred to 5 ml or 10 ml lyophilisation vials and pre-stopped with sterile rubber stoppers. The solution was then subjected to lyophilisation. The preparation of the composition, except during freeze drying, was performed at a temperature of between 2 and 10° C.

TABLE 1
Compositions of examples 1a to 1e.

| | Ex. 1a | Ex. 1b | Ex. 1c | Ex. 1d | Ex. 1e |
|---|---|---|---|---|---|
| Tigecycline | 50.0 | 50.0 | 50.0 | 50.0 | 50.0 |
| (mg/vial) | | | | | |
| Trehalose | | 100.0 | 50.0 | 50.0 | 50.0 |
| (mg/vial) | | | | | |
| Mannitol | | | 50.0 | 50.0 | 50.0 |
| (mg/vial) | | | | | |
| Arginine | | | | 17.4 | |
| (mg/vial) | | | | | |
| Histidine | | | | | 8.1 |
| (mg/vial) | | | | | |
| HCl 1N⁽¹⁾ | q.s. to pH | q.s. to pH | q.s. to pH | q.s. to pH | q.s. to pH |
| | 4.5-6.0 | 4.5-6.0 | 4.5-6.0 | 4.5-6.0 | 4.5-6.0 |
| Purified | q.s. to | q.s. to | q.s. to | q.s. to | q.s. to |
| Water⁽²⁾ | 2 ml | 2 ml | 2 ml | 2 ml | 2 ml |
| Total | 2 | 2 | 2 | 2 | 2 |
| (ml/vial) | | | | | |

⁽¹⁾“q.s. to pH” means the addition of acid in a quantity sufficient to bring the solution to a desired pH (e.g., q.s. to pH 4.5-6.0 means the addition of acid to bring the solution to a pH of 4.5-6.0).
⁽²⁾“q.s.” means adding a quantity sufficient to achieve a certain state (e.g., volume, i.e., to bring a solution to a desired volume).

The compositions of examples 1a to 1e were prepared at the same time and subjected to the same lyophilisation cycle in order to minimize external factors that could affect the final composition.

### Example 2

### Stability Studies for the Compositions Disclosed in Example 1

The compositions prepared according to Examples 1a to 1e were tested for stability at time zero after lyophilisation and also were stored in the lyophilised state at 40° C. and 75% of relative humidity (RH) for 9 days, before stability testing. Prior to testing, the lyophilised material was dissolved in a pharmaceutically acceptable reconstitution solution. The so obtained solutions were then analysed by HPLC according to standard methods well known to [symbol] the skilled person in the art. Table 2 indicates the impurity profile of the different compositions of example 1. The % of total impurities is indicated, as well as the % of tigecycline epimer, which is the major impurity.

TABLE 2
Impurity profile.

| | | % Total | | % Total |
|---|---|---|---|---|
| | % Epimer | Impurities | % Epimer | Impurities |
| | t = 0 | t = 0 | 9 days 40/75 | 9 days 40/75 |
| Ex. 1a | 1.61 | 2.88 | 2.14 | 3.20 |
| Ex. 1b | 0.86 | 1.13 | 1.65 | 1.99 |
| Ex. 1c | 0.90 | 1.33 | — | 2.37 |
| Ex. 1d | 1.08 | 1.76 | 1.76 | 2.59 |
| Ex. 1e | 1.14 | 1.86 | 1.83 | 2.73 |

### Example 3

### Preparation of Stable Tigecycline Compositions

The following tigecycline compositions were prepared as in example 1.

TABLE 3
Compositions of examples 3a and 3b.

| | Ex. 3a | Ex. 3b |
|---|---|---|
| Tigecycline (mg/vial) | 50.0 | 50.0 |
| Trehalose (mg/vial) | | 100.0 |
| Lactose (mg/vial) | 100.0 | |
| HCl 1N⁽¹⁾ | q.s. to pH 4.5-6.0 | q.s. to pH 4.5-6.0 |
| Purified Water⁽²⁾ | q.s. to 2 ml | q.s. to 2 ml |
| Total (ml/vial) | 2 | 2 |

⁽¹⁾“q.s. to pH” means the addition of acid in a quantity sufficient to bring the solution to a desired pH (e.g., q.s. to pH 4.5-6.0 means the addition of acid to bring the solution to a pH of 4.5-6.0).
⁽²⁾“q.s.” means adding a quantity sufficient to achieve a certain state (e.g., volume, i.e., to bring a solution to a desired volume).

The compositions of examples 3a and 3b were prepared at the same time and subjected to the same lyophilisation cycle in order to minimize external factors that could affect the final composition.

### Example 4

### Stability Studies for the Compositions Disclosed in Example 3

The compositions prepared according to Examples 3a and 3b were tested for stability at time zero after lyophilisation as described in example 2. Table 4 indicates the impurity profile of the compositions of example 3. The % of total impurities is indicated, as well as the % of tigecycline epimer, which is the major impurity.

TABLE 4
Impurity profile.

| | % Epimer | % Total Impurities |
|---|---|---|
| Ex. 3a | 0.89 | 1.10 |
| Ex. 3b | 0.88 | 1.08 |

### Example 5

### Water Content

The amount of water of the pharmaceutical compositions as herein disclosed was measured by Karl Fisher titration.

TABLE 5
Water content.

| | Ex. 1a | Ex. 1b | Ex. 1c | Ex. 1d | Ex. 1e |
|---|---|---|---|---|---|
| Water content (%) | 0.10 | 0.13 | 0.83 | 0.73 | 0.73 |

### Example 6

### Synthesis of Tigecycline and X-ray Powder Diffraction

Processes for the synthesis of tigecycline are described in U.S. Pat. No. 5,675,030, WO2006/130500, WO2006/130501 and WO2006/130431. To assess the crystallinity of tigecycline, XRD was performed. X-Ray Powder Diffraction was performed in a Bruker D8 Advance diffractometer with a θ0:2θ configuration and Bragg-Brentano geometry with a copper anode tube. The diffractogram is obtained for 2θ angles ranging from 3° to 70° with a step of 0.03° each second. The tube set-up is 40 kV and 30 mA, incident-beam divergence-limiting slit 12 mm, static sample, diffracted-beam receiving slit 0.2 mm and Nickel filter. The X-ray powder diffraction pattern (XRD) of the tigecycline used for the compositions described in example 1 and example 3 is shown in FIG. 2, which shows that the active agent is in amorphous form.

### Example 7

### Stable Tigecycline Formulations

| Qty per vial | Ex. 7a | | Ex. 7b | | Ex. 7c | |
|---|---|---|---|---|---|---|
| Tigecycline | 50 | mg | 50 | mg | 50 | mg |
| L-Arginine | 17.4 | mg | 25 | mg | 50 | mg |
| HCl⁽¹⁾ | q.s. to pH 4.5 | | q.s. to pH 4.5 | | q.s. to pH 4.5 | |
| Purified water⁽²⁾ | q.s. to 2 | ml | q.s. to 2 | ml | q.s. to 2 | ml |

⁽¹⁾“q.s. to pH” means the addition of acid in a quantity sufficient to bring the solution to a desired pH (e.g., q.s. for pH 4.5 means the addition of acid to bring the solution to a pH of 4.5).
⁽²⁾“q.s.” means adding a quantity sufficient to achieve a certain state (e.g., volume, i.e., to bring a solution to a desired volume).

### Example 8

### Batches Manufacture

Batches of 240 to 600 ml (120 to 300 vials) were prepared for each one of the examples 7a, 7b and 7c.

The L-arginine is weighed and added to about 80% of the purified water. The solutions are stirred for about 30 minutes to ensure complete dissolution of the arginine and 1 M hydrochloric acid is added until pH 4.5. Then, the tigecycline is weighed and added to the solution while stirring for 15 to 20 minutes. If necessary, 1 M hydrochloric acid is added until pH 4.5. Finally, purified water is added up to the desired volume. The purified water may be bubbled with nitrogen gas to reach an oxygen concentration in the water above 0.5 ppm and below 5 ppm, preferably between 0.6 and 2 ppm.

Once the solutions were ready, aliquots of 2 ml were prepared in 5 ml glass vials (Schott, VC005-20C, 5 mL Clear Type I Tubular Glass Vials). Then, rubber stoppers were placed and the vials were loaded into lyophiliser trays for lyophilisation. Orange color cakes were obtained in all the examples.

### Example 9

### Stability

After lyophilisation, the total impurity content and the epimer content of the compositions of each one of the examples 7a, 7b and 7c were analysed by HPLC at time zero and after 1, 2 and/or 3 months at 25° C. and 60% relative humidity (RH), 30° C. and 65% RH and 40° C. and 75% RH.

TABLE 6
Increase in total impurities (%) in the composition of
example 7a at 1 and 2 months in respect to time zero:

| Ex. 7a | 1 month | 2 months |
|---|---|---|
| 25° C./60% RH | 0.17 | 0.34 |
| 30° C./65% RH | 0.26 | 1.32 |
| 40° C./75% RH | 1.47 | 5.35 |

TABLE 7
Increase in epimer (%) in the composition of
example 7a at 1 and 2 months in respect to time zero:

| Ex. 7a | 1 month | 2 months |
|---|---|---|
| 25° C./60% RH | 0.00 | 0.35 |
| 30° C./65% RH | 0.15 | 1.16 |
| 40° C./75% RH | 1.31 | 5.25 |

TABLE 8
Increase in total impurities (%) in the composition of
example 7b at 1 and 2 months in respect to time zero:

| Ex. 7b | 1 month | 2 months |
|---|---|---|
| 25° C./60% RH | 0.32 | 0.33 |
| 30° C./65% RH | 0.89 | 0.70 |
| 40° C./75% RH | 1.14 | 1.44 |

TABLE 9
Increase in epimer (%) in the composition of
example 7b at 1 and 2 months in respect to time zero:

| Ex. 7b | 1 month | 2 months |
|---|---|---|
| 25° C./60% RH | 0.32 | 0.34 |
| 30° C./65% RH | 0.46 | 0.65 |
| 40° C./75% RH | 0.96 | 1.50 |

TABLE 10
Increase in total impurities (%) in the composition of
example 7c at 1, 2 and 3 months in respect to time zero:

| Ex. 7c | 1 month | 2 months | 3 months |
|---|---|---|---|
| 25° C./60% RH | 0.00 | 0.13 | 0.21 |
| 30° C./65% RH | 0.00 | 0.33 | 0.03 |
| 40° C./75% RH | 0.24 | 0.85 | 0.67 |

TABLE 11
Increase in epimer (%) in the composition of
example 7c at 1, 2 and 3 months in respect to time zero:

| Ex. 7c | 1 month | 2 months | 3 months |
|---|---|---|---|
| 25° C./60% RH | 0.00 | 0.00 | 0.02 |
| 30° C./65% RH | 0.00 | 0.26 | 0.00 |
| 40° C./75% RH | 0.18 | 0.74 | 0.44 |

### Example 10

### Water Content

The amount of water of the pharmaceutical compositions as herein disclosed was measured by Karl Fisher titration.

TABLE 12
Percent water content in the composition of example 7a:

| Ex. 7a | t = 0 | t = 1 month | t = 2 months | t = 3 months |
|---|---|---|---|---|
| 25° C./60% RH | 0.59 | 1.46 | 1.12 | 2.43 |
| 30° C./65% RH | 0.59 | 1.10 | 1.34 | 1.58 |
| 40° C./75% RH | 0.59 | 1.47 | 1.65 | 1.77 |

TABLE 13
Percent water content in the composition of example 7b:

| Ex. 7b | t = 0 | t = 1 month | t = 2 months |
|---|---|---|---|
| 25° C./60% RH | 0.21 | 0.44 | 0.47 |
| 30° C./65% RH | 0.21 | 0.38 | 0.66 |
| 40° C./75% RH | 0.21 | 0.68 | 0.84 |

TABLE 14
Percent water content in the composition of example 7c:

| Ex. 7c | t = 0 | t = 1 month | t = 2 months | t = 3 months |
|---|---|---|---|---|
| 25° C./60% RH | 0.45 | 0.62 | 0.84 | 1.24 |
| 30° C./65% RH | 0.45 | 0.71 | 0.94 | 1.00 |
| 40° C./75% RH | 0.45 | 0.95 | 1.22 | 1.23 |

### Example 11

### Freeze-drying Process

The temperature of collapse in the freeze drying of the aqueous solutions of tigecycline and an excipient were analysed. It was found for example that the temperature of collapse of the pharmaceutical composition of example 3a was −29° C. and that of example 7c was −26° C. First, longer lyo cycles with very low primary drying temperatures were used to keep the product always under the temperature of collapse. Surprisingly, even having such low collapse temperatures, the best freeze drying cycles were those with a higher primary drying temperature, set at between −20° C. and 10° C. Particularly for the composition of example 3a, the best results were obtained with a primary drying temperature of about 0° C., and for the composition of example 7c, the best results were obtained with a primary drying temperature of about −10° C. For the pharmaceutical composition of example 3a, the best cakes were those freeze dried with a primary drying temperature of about 0° C. and a secondary drying temperature of about 30° C. For the pharmaceutical composition of example 7c, the best cakes were those freeze dried with a primary drying temperature of about −10° C. and a secondary drying temperature of about 30° C.

The freeze drying of the pharmaceutical compositions of the examples was performed at different temperatures, pressures and durations and it was found that good cakes with no collapse or cavities were obtained when the freeze drying took at least 16 hours and less than 42 hours, from the start of the freezing of the solution to the end of the secondary drying. Also, very good cakes were obtained when the solution was frozen at a temperature below −20° C. and when the primary drying was set at a temperature between −20 and 10° C., and was maintained for at least 8 or 10 hours. Very good cakes were obtained when the secondary drying temperature was set between 20 and 40° C., and maintained for at least 5 hours.

1. A cardboard box with a patient information leaflet comprising at least one unit of a pharmaceutical composition comprising an active agent and arginine or a pharmaceutically acceptable salt thereof, wherein the active agent is a glycylcycline or a pharmaceutically acceptable salt thereof and wherein the weight ratio active agent:arginine is between 2:3 and 3:1 or at least one vial tubular glass vial comprising said pharmaceutical composition.

2. (canceled)

3. The cardboard box according to claim 1, wherein the weight ratio is about 1:1 .

4. (canceled)

5. The cardboard box according to claim 1, wherein the pharmaceutical composition comprises between 45 and 55 mg of active agent by unit dose.

6-7. (canceled).

8. The cardboard box according to claim 1, wherein the active agent is tigecycline or a pharmaceutically acceptable salt thereof.

9-10. (canceled)

11. The cardboard box according to claim 1, wherein the active agent is in amorphous form and arginine is L-arginine.

12. (canceled)

13. The cardboard box according to claim 12, further comprising a pH modifying agent or a mixture of pH modifying agents, and wherein the pH modifying agent is an acid, a buffer, or mixtures. thereof.

14. The cardboard box according to claim 13, wherein the acid is hydrochloric acid.

15-17. (canceled)

18. The cardboard box according to claim 115, solution is an aqueous solution wherein the pH of the solution is between 3.5 and 7.0.

19. The cardboard box according to claim 18, wherein the pH of the composition is between 4.0 and 5.0.

20-22. (canceled)

23. The cardboard box according to claim 1, wherein the composition is in the form of a lyophilised cake or powder and wherein the water content of the lyophilised cake or powder is below 3% by weight of the total amount of the lyophilised cake or powder.

24. (canceled)

25. The cardboard box according to claim 1, wherein the pharmaceuti cal composition comprises lyophilised cake or lyophilised powder obtainable by lyophilisation, and wherein said lyophilised cake or powder is suitable for reconstitution to form a liquid composition for parenteral, or intravenous administration.

26-28. (canceled)

29. A process for the manufacture of a pharmaceutical composition as defined in claim 1 the process comprising at least one of the following steps:
i) preparing an aqueous solution of arginine in purified water;
ii) optionally adjusting the pH of the solution prepared in step (i) to between 3.0 to 8.0;
iii) dissolving the active agent in the solution obtained in step (i) or step (ii);
iv) optionally adjusting the pH of the solution prepared in step (iii) to between 3.0 to 8.0;
v) optionally filtering the solution obtained in step (iii) or in step (iv);
vi) optionally freezing the solution obtained in step (iii), (iv) or (v); and
vii) optionally freeze drying the frozen solution obtained in step (vi).

30. The process according to claim 29,
wherein nitrogen is bubbled in the solution.

31-32. (canceled)

33. A process for the manufacture of a lyophilised cake or powder comprising a glycylcycline or a pharmaceutically acceptable salt thereof as active agent, and at least one pharmaceutically acceptable excipient, wherein said process comprises:
freeze drying a solution comprising a glycylcycline or a pharmaceutically acceptable salt thereof as active agent and at least one pharmaceutically acceptable excipient,
wherein said freeze drying comprises the steps of
freezing the solution,
primary drying and secondary drying, and
wherein the freeze drying takes at least 16 hours and less than 72 hours from the start of the freezing of the solution to the end of the secondary drying.

34. The process according to claim 33, wherein the freeze drying comprises the following steps:
i) freezing the solution at a temperature below −20° C. and maintaining said temperature for at least 1 hour;
ii) primary drying the frozen solution of step (i) under vacuum and at a temperature between −20 and 10° C., and maintaining said conditions for at least 10 hours; and
iii) secondary drying the primary dried frozen solution of step (ii) under vacuum and at a temperature between 20 and 40° C., and maintaining said conditions for at least 5 hours.

35. The process according to claim 34,
wherein the temperature in step (i) is between −55 and −35° C maintained at least 2 hours and/or p1 wherein the temperature in step (ii) is between −15 and 10° C., maintained at least 12 hours; and/or
wherein the temperature in step (iii) is between 25 and 35° C., maintained at least 7 hours.

36-37. (canceled)

38. The process according to claim 29,
wherein the process comprises the freezing of step (vi) that occurs for at least 10 hours under reduced ressure of from 0.01 to 1 mbar ata temperature between −20 and 10′C: and
wherein the process comprises the freeze drying of step (vii) that occurs for at least 5 hours under reduced aressure of from 0.001 to 1 mbar at a temperature between 20 and 40° C.

39. A pharmaceutical composition obtained by the process according to claim 29.

40. A pharmaceutical batch comprising at least 1,200 units of the pharmaceutical composition as defined in claim 1.

41-44. (canceled)

45. A cardboard box comprising the pharmaceutical composition as defined in claim 39.
